(19) United States
(12) Patent Application Publication
(10) Pub. No.: US 2025/0161100 A1
(43) Pub. Date: May 22, 2025

(54) Breath Improver

(71) Applicant: Jianhua FAN, Nashville, TN (US)

(72) Inventor: Jianhua FAN, Nashville, TN (US)

(21) Appl. No.: 18/445,605

(22) Filed: Nov. 16, 2023

(51) Int. Cl. A61F 5/56

(52) U.S. Cl. CPC A61F 5/566

(57) ABSTRACT

A pipe for mouth breath having two portions: inner portion and out portion, at least one hole disposed on the wall of the inner portion, the pipe is open ends, while in use, the pipe been placed in user's mouth between teeth and cheek, the out portion passed through lips and open to air. Inhaling: air is taken into the out portion, gets out of the inner portion, passed through gaps between teeth and into mouth chamber. Exhaling: air gets out of mouth by reversed process of inhaling. Also, the pipe can made into “U” shaped, two ends of the pipe became inner portions, at least one short pipe attached on the middle portion, or the middle portion been enlarged for air gets in and out of mouth or cut to open between inner portion and middle portion, the inner portion can be extended long enough to push middle portion out of mouth and open to air. Also again, a dental retainer may be used, middle of one pipe joined one end of the dental retainer, middle of another pipe joined another end of the dental retainer, the low part of the dental retainer may be deviated and forwarded from the up part for special use.

### BACKGROUND OF THE INVENTION

### Field of the Invention

The present invention relates to a breath improvement apparatus, more particularly to a mouth breath improvement apparatus for people at sleep.

### Description of the Prior Art

One existed device for breath improvement is called “ZQuiet”, in which the device pushes teeth to let mouth open. Clearly, that made mouth open by force, so that user usually feels uncomfortable.

It can be seen then that there is a need for new devices to improve breath quality and make the user feel comfortable.

### SUMMARY OF THE INVENTION

The present invention relates to a breath improvement apparatus, more particularly to a mouth breath improvement apparatus, or said mouth breath improver.

The present invention discloses a mouth breath improver, the apparatus is improving sleep quality, reducing snoring, apneas and other problems.

In one embodiment of the present invention, a breath improver includes: a pipe for mouth breath, the pipe having two portions, inner portion and out portion, at least one hole disposed on the wall of the inner portion, while in use, the inner portion been placed between teeth and cheek, the length of the inner portion may be varied, the out portion passed through lips and open to air. Inhaling, air gets into the out portion, then gets out of the hole(s) and open end of the inner portion, then passed through gaps between teeth and into mouth chamber. Exhaling, air in mouth chamber passed through gaps between teeth, then gets into the inner portion of the pipe, then gets out of mouth through out portion of the pipe, two pipes may be used for each side of mouth. So that, people do not need to push their mouth opened while doing mouth breath.

In second embodiment of the present invention, a breath improver includes: a pipe, the pipe has three portions, two inner portions and middle portion, the pipe been made in “U” shaped, the “U” shaped pipe can be adjusted to match user's tooth arrangement shape, multiple holes disposed on the wall, an air intake pipe connected to the middle portion, while in use, the air intake pipe passed through lips and opened to air.

Still in second embodiment of the present invention, two air intake pipes disposed on the middle portion of the “U” shaped pipe instead of one air intake pipe.

Still in second embodiment of the present invention, the middle portion of the “U” shaped pipe been enlarged to push lips open instead of air intake pipe(s).

Still in second embodiment of the present invention, the two inner portions been prolonged, while in use, the middle portion been extended to outside of mouth.

Still in second embodiment of the present invention, between the inner portion and the middle portion of the “U” shaped pipe been cut to open.

In third embodiment of the present invention, a breath improver includes: two pipes and one dental retainer, at least one hole disposed on the inner portion, middle of one pipe connected to one end of the dental retainer, middle of another pipe connected to another end of the dental retainer, the dental retainer can be adjusted to matched user's tooth arrangement shape. For a special use, such as to reduce snoring, apneas, and other problems, the lower part of the dental retainer may be moved forward a little than up part.

These and various other advantages and features of novelty which characterize the invention are particularly pointed out in the claims annexed hereto and form a part hereof, for a better understanding of the invention, its advantages, and the objects obtained by its use, reference should be made to the drawings which form a further part hereof, and to the accompanying descriptive matter, in which there are illustrated and described specific examples of an apparatus in accordance with the invention.

### BRIEF DESCRIPTION OF THE DRAWINGS 4

Referring now to the drawings in which like reference numbers represent corresponding parts throughout.

FIG. 1 is a perspective view of one breath improver in accordance with the principles of the present invention.

FIG. 2 is a perspective view of second breath improver in accordance with the principles of the present invention.

FIG. 3 is a side view of FIG. 1 while it's in use.

FIG. 4 is an A-A crosscut view of FIG. 2 while it's in use.

FIG. 5 is another perspective view of FIG. 2 in which one air intake pipe only.

FIG. 6 is a third perspective view of FIG. 2 in which the middle portion is enlarged.

FIG. 7 is a side view of FIG. 6 while in use.

FIG. 8 is a crosscut view of an extended “U” shaped breath improver.

FIG. 9 is a perspective view of third breath improver in accordance with the principles of the present invention.

FIG. 10 are B-B crosscut views of the dental retainer in FIG. 9.

FIG. 11 is a perspective view of “U” shaped pipe in which between inner portion and middle portion is cut to open.

FIG. 12 is a perspective of FIG. 11 in which the whole middle portion is cut to open.

### DETAILED DESCRIPTION OF THE PREFERRED EMBODIMENTS

In the following description of the exemplary embodiment, reference is made to the accompanying drawings which form a part hereof, and in which it is shown by way of illustration the specific embodiment in which the invention may be practiced. It is to be understood that other embodiments may be utilized as structural changes may be made without departing from the scope of the present invention.

The present invention provides a breath improver apparatus. The breath improver apparatus in accordance with the principles of the present invention is capable of being improved mouth breath without force to push mouth opened and is capable of being increased sleep quality, reduced snoring, apneas, and other problems.

In FIG. 1, there is generally illustrated by reference numeral 2 one embodiment of a breath improver apparatus in accordance with the principles of the present invention. The breath improver apparatus numeral 2 includes an inner portion 11 and out portion 12, the portions are made by pipe, the size, shape, and length of the pipe may be varied, at least one hole numeral 5 disposed on the wall of the inner portion. While in use, the inner portion is seated between teeth and cheek, the out portion passed through lips and opened to air. Inhaling, air gets into the out portion, then gets out of the inner portion through open end and the at least one hole on the wall, then the air passed through gaps between teeth and gets into mouth chamber. Exhaling, air gets out of the mouth by reversed process of inhaling.

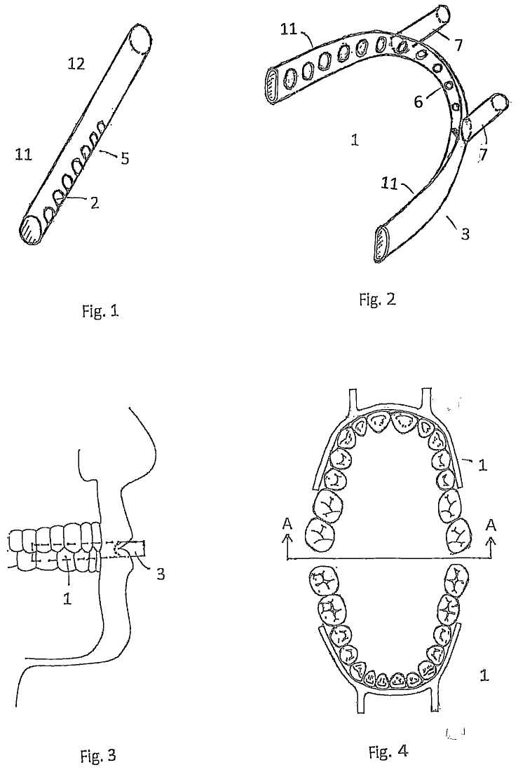

In FIG. 2, there is generally illustrated by reference numeral 1 second embodiment of the breath improver apparatus in accordance with the principles of the present invention. The breath improver apparatus numeral 1 includes a “U” shaped pipe 3 and air intake pipe 7, the “U” shaped pipe 3 includes three portions: 2 inner portions 11 and middle portion 6, at least one hole 5 disposed on the wall of each portion, air intake pipes 7 connected to the “U” shaped pipe, the size, shape, and length of the “U” shaped pipe and air intake pipes may be varied, the “U” shaped pipe can be adjusted to match user's tooth arrangement shape, air intake pipes passed through lips and open to air while in use.

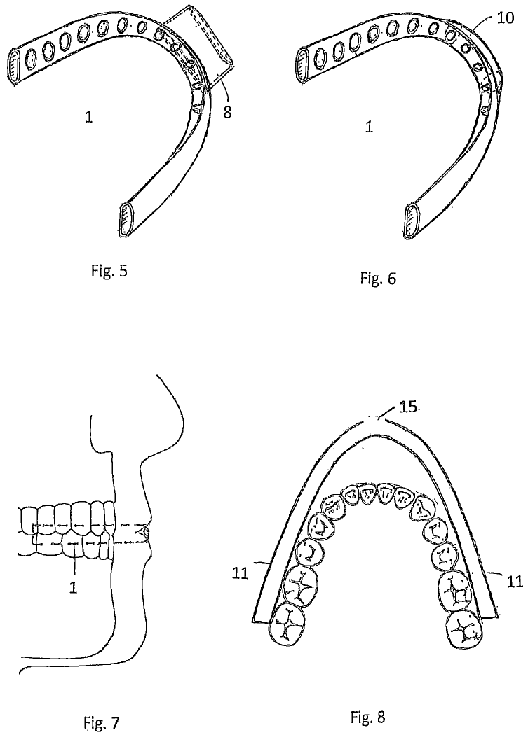

FIG. 5 illustrates an air intake pipe numeral 8 connected to the middle portion of the “U” shaped pipe.

FIG. 6 illustrates an air intake portion numeral 10, in which the air intake portion 10 is formed by enlarging the middle portion of the “U” shaped pipe.

FIG. 8 illustrates a new “U” shaped pipe numeral 21, the new “U” shaped pipe formed from “U” shaped pipe numeral 3 in FIG. 2, in which the inner portions 11 extended long enough to let middle portion 6 pass lips and into air while in use.

FIGS. 11 and 12 illustrate other “U” shaped pipes, in which the “U” shaped pipe 3 in FIG. 2 being cut to open between the inner portion and middle portion of the pipe, so that air can get in and out of the pipe through the cut point, at least one hole disposed on the middle portion in FIG. 11, the middle portion cut to open air numeral 18 in FIG. 12.

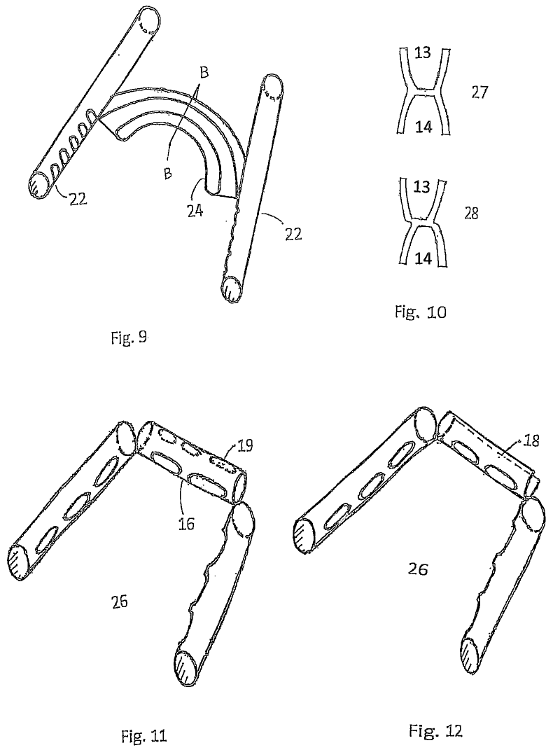

In FIG. 9, there is generally illustrates by reference numeral 20 third embodiment of the breath improver apparatus in accordance with the principles of the present invention. The breath improver apparatus numeral 20 includes two breath improver numeral 2 of FIG. 1 and a dental retainer numeral 24, middle of one breath improver numeral 2 joined one end numeral 22 of the dental retainer 24, middle of another breath improver numeral 2 joined another end numeral 29 of the dental retainer numeral 24, the dental retainer can be adjusted to match user's tooth arrangement shape.

FIG. 10 shown a B-B crosscut of the dental retainer 24 in FIG. 9, In side view of the B-B crosscut numeral 28, there is a disparity between the up portion and the low portion of the dental retainer, it is designed to push low jaw forward and avoid blocking up throat airway, so that snoring, apneas and other problems possible been reduced.

The foregoing description of the exemplary embodiment of the invention has been presented for the purposes of illustration and description. It is not intended to be exhaustive or to limit the invention to the precise form disclosed. Many modifications and variations are possible in light of the above teaching. It is intended that the scope of the invention be limited not with this detailed description, but rather by the claims appended hereto.

1. A breath improver apparatus, comprising:
a pipe for mouth breath having two portions, inner portion and out portion, at least one hole disposed on the wall of inner portion, the pipe is open ends.

2. A breath improver apparatus, comprising:
two pipes, each pipe having two portions, inner portion and out portion, at least one hole disposed on the wall of the inner portion, the pipes are open ends;
a dental retainer, middle of one pipe joined one end of the dental retainer, middle of another pipe joined another end of the dental retainer.

3. The breath improver apparatus of claim 2, wherein between the up part and the lower part of the dental retainer without deviation.

4. The breath improver apparatus of claim 2, wherein the lower part of the dental retainer deviated and forwarded from the up part of the dental retainer.

5. A breath improver apparatus, comprising:
a “U” shaped pipe, the “U” shaped pipe having three portions, two inner portions and one middle portion, at least one hole disposed on the wall of each portion, the pipe is open ends, at least one air intake pipe is attached on the middle portion.

6. The breath improver apparatus of claim 5, wherein the “U” shaped pipe can be adjusted.

7. The breath improver apparatus of claim 5, wherein the air intake pipe can be replaced by enlarging the pipe of the middle portion.

8. The breath improver apparatus of claim 5, wherein the “U” shaped pipe can be cut to open between the middle portion and the inner portion for air in and out of the pipe.

9. The breath improver apparatus of claim 5, wherein the inner portions can be extended longer enough to let the middle portion open to air while in use.
